(19) United States
(12) Patent Application Publication
(10) Pub. No.: US 2023/0030647 A1
(43) Pub. Date: Feb. 2, 2023

(54) COMPOSITION FOR IMPROVING SKIN PROPERTIES AND METHOD FOR IMPROVING SKIN PROPERTIES

(71) Applicant: Smart Lab Inc., Tokyo (JP)

(72) Inventors: Yasumitsu SAKAI, Tokyo (JP); Michihiro TAMUNE, Tokyo (JP)

(21) Appl. No.: 17/873,141

(22) Filed: Jul. 26, 2022

(30) Foreign Application Priority Data
Jul. 28, 2021 (JP) 2021-123116
Jul. 5, 2022 (JP) 2022-108655

(51) Int. Cl. A61K 8/73; A61K 8/60; A61K 8/9789; A61K 8/99

(52) U.S. Cl. CPC A61K 8/732; A61K 8/60; A61K 8/99; A61K 8/9789

(57) ABSTRACT

A composition for improving skin properties and a method for improving skin properties are provided. The composition containing a high-amylose cornstarch for improving skin properties is provided. The method for improving skin properties including a step of taking a high-amylose cornstarch by a daily amount effective for improving the skin properties for four weeks or more is provided. The method excludes a medical treatment to human.

### CROSS-REFERENCES TO RELATED APPLICATIONS

This patent specification is based on Japanese patent application, No. 2021-123116 filed on Jul. 28, 2021 in the Japan Patent Office and Japanese patent application, No. 2022-108655 filed on Jul. 5, 2022 in the Japan Patent Office, the entire contents of which are incorporated by reference herein.

### BACKGROUND OF THE INVENTION

### 1. Field of the Invention

The present invention relates to a composition for improving skin properties and a method for improving skin properties.

### BACKGROUND ART

### 2. Description of Related Art

A skin is the largest organ covering the whole human body. The skin separates an inside body from an external environment and plays an important role for maintaining homeostasis of a living body. An epidermis, which is an outermost layer, is a thin membrane having an average thickness of approximately 0.2 mm. The epidermis has a role of preventing transpiration of moisture and a role of a barrier for preventing infiltration of harmful components from the outside.

A fibroblast, which is present in dermis, produces extracellular matrix such as collagen, elastin and hyaluronic acid to maintain skin structure. The collagen and the elastin, which are fibrous protein, form a network in a meshed state to keep elasticity and tension of the skin. The fibroblast encourages turnover where decomposition and regeneration of fibrous components are gradually repeated. Thus, an activated state of the fibroblast has a material effect on elasticity and tension of the skin. It is considered that health of the skin is maintained by the above described function of the epidermis and the dermis.

However, when the extracellular matrix such as the collagen, the elastin and hyaluronic acid of the dermis is reduced by the damage from an external environment such as ultraviolet light and dryness or by the reduction of the fibroblast and deterioration of activities caused by aging, a barrier function of the skin is deteriorated, moisture and elasticity are lost, and aging of the skin is caused. Wrinkle and flabbiness are caused not only by the reduction of the production of the collagen and the elastin but also by the decomposition of the collagen, for example.

Accordingly, it is required to improve the skin properties.

Regarding the technology related to the above described problem, the purpose of Patent document 1 is to provide a new composition for improving skin properties, a skin improving agent and a skin improving method, for example. Patent document 1 provides the composition for improving skin properties and the skin improving agent containing a luminacoid derived from cacao beans and a luminacoid derived from almond. Patent document 1 also discloses to provide the skin improving method including a step of taking the luminacoid derived from cacao beans and the luminacoid derived from almond by a daily amount effective for improving the skin properties for four weeks or more.

[Patent document 1] Japanese Unexamined Patent Application Publication No. 2018-100263

### BRIEF SUMMARY OF THE INVENTION

The present invention aims for providing a composition for improving skin properties and a method for improving skin properties.

The inventors of the present invention found that the composition containing high-amylose cornstarch improved skin properties of human. The present invention is based on the above described knowledge.

For achieving the above described purpose, the summary of the present invention is as follows.
[1] a composition containing a high-amylose cornstarch for improving skin properties
[2] the composition of [1] further containing an inulin
[3] the composition of [1] or [2] further containing a fructo-oligosaccharide
[4] the composition of [2] or [3] further containing a β-glucan
[5] the composition of [4] further containing a lactic acid bacterium
[6] the composition of any one of [1] to [5] further containing a soybean
[7] the composition of any one of [1] to [6] wherein the high-amylose cornstarch contains 60% or more of a moist heat-treated resistant starch
[8] the composition of any one of [1] to [7] wherein an amount of the composition is 15 g or more
[9] the composition of any one of [1] to [9] wherein an amount of the composition is four weeks' worth or more
[10] a method for improving skin properties including a step of taking a high-amylose cornstarch by a daily amount effective for improving the skin properties for four weeks or more (excluding a medical treatment to human

When the composition of the present invention is taken or the method for improving skin properties is used, the effect of improving skin properties can be obtained.

### BRIEF DESCRIPTION OF THE DRAWINGS

FIG. 1 is a graph showing a test result of eight examinees taking (1) whey protein.

FIG. 2 is a graph showing a test result of eight examinees taking (2) water.

FIG. 3 is a graph showing a test result of eight examinees taking (3) a composition containing only high-amylose cornstarch.

FIG. 4 is a graph showing a test result of eight examinees taking (4) a composition containing high-amylose cornstarch and inulin.

FIG. 5 is a graph showing a test result of eight examinees taking (5) a composition containing six kinds of components.

### DETAILED DESCRIPTION OF THE INVENTION

The present invention is a composition for improving skin properties. The composition is taken by human and nonhuman animal. The composition contains a high-amylose cornstarch. Here, the high-amylose cornstarch is a resistant starch (hardly digestible starch) derived from corn. The type of the resistant starch is RS2b which is a starch containing a high content of amylose.

The composition can further contain an inulin. In this case, the components of the composition are the combination of the high-amylose cornstarch and the inulin.

The composition can further contain a fructo-oligosaccharide. In this case, the components of the composition are the combination of the high-amylose cornstarch and the fructo-oligosaccharide or the combination of the high-amylose cornstarch, the inulin and the fructo-oligosaccharide.

The composition can further contain a β-glucan. In this case, the components of the composition are the combination of the high-amylose cornstarch, the inulin and the β-glucan, the combination of the high-amylose cornstarch, the fructo-oligosaccharide and the β-glucan or the combination of the high-amylose cornstarch, the inulin, the fructo-oligosaccharide and the β-glucan.

As for the β-glucan, the β-glucan derived from bread yeast is superior in terms of cost.

The composition can further contain a lactic acid bacterium. In this case, the components of the composition are the combination of the high-amylose cornstarch, the inulin, the β-glucan and the lactic acid bacterium, the combination of the high-amylose cornstarch, the fructo-oligosaccharide, the β-glucan and the lactic acid bacterium or the combination of the high-amylose cornstarch, the inulin, the fructo-oligosaccharide, the β-glucan and the lactic acid bacterium.

As for the lactic acid bacterium, sporogenous lactic acid bacterium is preferable.

The composition can further contain a soybean. The soybean is formed in a powder. The soybean formed in a paste can be also used. In this case, the components of the composition are the combination of the high-amylose cornstarch and the soybean, the combination of the high-amylose cornstarch, the inulin and the soybean, the combination of the high-amylose cornstarch, the fructo-oligosaccharide and the soybean, the combination of the high-amylose cornstarch, the inulin, the fructo-oligosaccharide and the soybean, the combination of the high-amylose cornstarch, the inulin, the β-glucan and the soybean, the combination of the high-amylose cornstarch, the fructo-oligosaccharide, the β-glucan and the soybean, the combination of the high-amylose cornstarch, the inulin, the fructo-oligosaccharide, the β-glucan, the soybean, the high-amylose cornstarch, the inulin, the β-glucan, the lactic acid bacterium and the soybean, the combination of the high-amylose cornstarch, the fructo-oligosaccharide, the β-glucan, the lactic acid bacterium and the soybean or the combination of the high-amylose cornstarch, the inulin, the fructo-oligosaccharide, the β -glucan, the lactic acid bacterium and the soybean.

The high-amylose cornstarch contained in the composition can contain 60% or more of a moist heat-treated resistant starch.

The composition used in the following explanation is one of the above described compositions.

In the high-amylose cornstarch or the composition also containing the other components than the high-amylose cornstarch, it is preferable that the high-amylose cornstarch is the main composition. In that case, the content of the high-amylose cornstarch is preferably 50% or more. It is more preferable that the content of the high-amylose cornstarch is 75% or more.

In particular, in the composition formed of a plurality of components, plural kinds of luminacoids having different molecular sizes are combined. Consequently, a time lag of fermentation of intestinal flora is produced in a large intestine. Thus, the composition can be spread throughout the large intestine and short-chain fatty acid can be produced in the entire large intestine.

Here, the improvement of skin properties means the improvement of a moisture content of epidermal stratum corneum, the improvement of skin turnover, the improvement of skin inflammation and/or the improvement of subjective symptom related to skin.

The composition can be provided in the form of medicine, quasi-drug, food and beverage, feed or the like.

It is preferable to contain 15 g or more composition per intake. It is preferable to take the composition three times per day before meal. It is preferable to drink the composition after dissolving it in 200 cc of water (including hot water).

It is not necessary to take the composition three times per day. It is also possible to take the composition less than three times per day or more than three times per day. It is preferable to take the composition every day. It is not necessary to take the composition before meal. It is also possible to take the composition after meal, between meals, at the time of awakening, before bed, at a predetermined time interval or at a predetermined time (e.g., at 8 a.m., 1 p.m. and 8 p.m.), for example. It is not necessary to take the composition with 200 cc of water. It is also possible to take the composition with more than 200 cc of water or less than 200 cc of water. It is also possible to take the composition with the liquid other than the water (e.g., milk and juice). It is also possible to provide a food containing the composition as raw material (i.e., the composition is mixed with the food). When the composition is orally taken by the human and nonhuman animal, the condition of the composition can be arbitrarily selected from a normal temperature, a warm state and a cold state. However, it should be considered that the visceral function may be deteriorated if the temperature of the intestine is lowered when the composition is taken.

As for the method for taking the composition, it is general to orally take the composition by the human and nonhuman (oral intake). It is not necessary to orally take the composition. Other than the oral intake, it is also possible to take the composition into the body by a tube administration, a nasal tube administration, a suppository and the like in accordance with the shape of the composition or the agent for improving the skin properties. For example, when the composition is a liquid state having viscosity or a semisolid state, the composition can be administered to the human and nonhuman animal even if the oral intake is impossible.

As for the type of the oral agent for the oral intake, granule, powder, tablet (including sugar-coated tablet), pill, capsule, syrup, emulsion, suspension and the like are possible. The above described composition is preferably formed only of the components of the composition. It is preferable that so-called additives are not contained. However, the composition can be formulated into a pharmaceutical preparation using the pharmaceutically acceptable carrier by the method ordinarily used in the art. As for the pharmaceutically acceptable carrier, excipient, binder, diluent, additive, perfume, buffer, thickener, colorant, stabilizer, emulsifier, dispersant, suspension, antiseptic and the like are possible.

The skin properties can be improved by taking the composition for four weeks (28 days) or more. Accordingly, it is possible to provide the composition used for four weeks as a set.

The present invention is a method for improving skin properties including a step of taking a high-amylose cornstarch by a daily amount effective for improving the skin properties for four weeks or more.

However, a medical treatment to human is excluded.

As for the method for improving skin properties, the high-amylose cornstarch can be replaced with the above described composition (combination of the high-amylose cornstarch, the inulin, the fructo-oligosaccharide, the β-glucan, the lactic acid bacterium and the soybean).

### Examples

Although the present invention will be explained based on the following examples, the present invention is not limited to the following examples.

The following examinations were conducted for verifying the influence on the skin regarding (1) the composition containing only the high-amylose cornstarch, (2) the composition containing the high-amylose cornstarch and the inulin and (3) the composition containing six components (the high-amylose cornstarch, the inulin, the fructo-oligosaccharide, the β-glucan, the lactic acid bacterium and the soybean).

As for the test method, double blind test (DBT) was used.

As for the measuring instrument, Corneometer CM825 (model number: CM825MP) manufactured by Courage+Khazaka electronic GmbH (Germany) was used.

As for the measuring part, one part was measured at the center of the forehead above between the eyebrows of the face. The “moisture content” contained in a part (mainly stratum corneum) approximately 15 µm inside from the skin surface was measured twelve times and the average value was calculated from the values of ten times excluding the maximum value and the minimum value.

The measurement was conducted for totally 30 examinees (8 adults, 5 groups) in a constant-temperature and constant-humidity room (24° C., 30%Rh). The examinees waited in a state of being exposed for 30 minutes or more and then the measurement was started.

The measurement period was started from May 1, 2021, 28 days later and 56 days later by dividing the examinees into the following five groups in accordance with the substances to be taken. The measurement was conducted in the time zone of 10 a.m. to 12 a.m..
(1) whey protein (protein: 92.4%, water: 4.0%, fat: 1.3%, ash: 2.3%)
(2) water
(3) high-amylose cornstarch (containing 60% or more of moist heat-treated resistant starch)
(4) composition containing high-amylose cornstarch (containing 60% or more of moist heat-treated resistant starch) and inulin
(5) composition containing six kinds of components (containing high-amylose cornstarch, inulin, soybean, fructo-oligosaccharide, β-glucan derived from bread yeast, sporogenous lactic acid bacterium)

Similar to (3), the high-amylose cornstarch contained in the composition (5) contains 60% or more of moist heat-treated resistant starch.

(1) and (2) correspond to placebo drugs. (3), (4) and (5) correspond to an embodiment of the present invention.

As for (1), (3), (4) and (5), 15 g of powder was dissolved in 200 cc of water and taken by the examinees three times per day before meal. As for (2), 200 cc of water was taken by the examinees three times per day before meal.

Note that the examinees are screened before the main examination in the following conditions.

The questionnaires were carried out about the lifestyle (e.g., age, existence/absence of anamnesis/complication/alimentary allergy, ingestion state of medicine/ health food, state of alcohol drinking) and the self-awareness (e.g., condition of skin, state of bowels). In addition, the physical test (e.g., body height, weight, body weight, blood pressure, pulse rate, BMI) was carried out. The exclusion criteria (details of the exclusion criteria are shown below) were applied to select 32 adults.

The exclusion criteria in the screening are as follows.

The following examinees were eliminated from the object to be screened.
The examinee who currently has some kinds of chronic diseases and receives medicinal treatment
The examinee who is allergic to milk or soybean
The examinee who has lactose intolerance
The examinee who had strong emotional stress apparently different from a usual time within three months before the screening
The examinee who changed eating habits to the one apparently different from a usual time within three months before the screening
The examinee who drank medicine or health food apparently different from a usual time within three months before the screening
The examinee who joined in the other clinical trials or monitor tests within three months before the screening or the examinee who is planning to join the other clinical trials after the examinee agrees with the main examination
The examinee who is pregnant or the examinee who is planning or desiring to be pregnant within the test period
The examinee who is currently breastfeeding
The examinee who has an excessive alcohol intake (drinking 60 g or more alcohol per day on average in terms of pure alcohol)

Note that the excessive alcohol intake (high risk group) corresponds to the person who drinks 60 g or more alcohol per day in a drinking day in terms of pure alcohol (according to the survey of the research group in Ministry of Health, Labor and Welfare in 2013). - The examinee who is judged to be inadequate because of the other reasons

In addition, the examinee who newly took the medicine or health food different from a usual time during the examination was eliminated after the examination.

(1) Table 1 shows the measurement results of eight examinees taking the whey protein. The results measured by using the above described measuring instrument before the intake, 28 days after the intake and 56 days after the intake are shown. Namely, the value (average value of ten times of measurement) indicating the moisture content contained in a part approximately 15 µm inside from the skin surface of the forehead is shown.

Table 1

forehead average value

| (1) whey protein | before intake | after 28 days | after 56 days |
|---|---|---|---|
| 1 | 46.99 | 47.67 | 46.33 |
| 2 | 55.24 | 54.94 | 54.37 |
| 3 | 56.49 | 55.73 | 56.17 |
| 4 | 56.75 | 57.19 | 56.90 |
| 5 | 62.07 | 60.04 | 58.68 |
| 6 | 62.58 | 62.03 | 62.41 |
| 7 | 62.32 | 62.37 | 62.60 |
| 8 | 63.40 | 65.97 | 65.00 |

Table 2 shows the quartiles of the test result. The quartiles are obtained by arranging the data in an ascending order (from the smallest) and equally dividing the data into four. The value arranged at the position corresponding to one fourth of the total number from the smallest is the first quartile, the value arranged at the center is the second quartile (i.e., center value), and the value arranged at the position corresponding to three fourth is the third quartile.

Table 2

forehead average value

| (1) whey protein | before intake | after 28 days | after 56 days |
|---|---|---|---|
| highest value | 63.40 | 65.97 | 65.00 |
| third quartile | 62.45 | 62.20 | 62.51 |
| second quartile | 59.41 | 58.62 | 57.79 |
| first quartile | 55.87 | 55.34 | 55.27 |
| lowest value | 46.99 | 47.67 | 46.33 |

(2) Table 3 shows the measurement results of eight examinees taking the water. The results measured by using the above described measuring instrument before the intake, 28 days after the intake and 56 days after the intake are shown. Namely, the value (average value of ten times of measurement) indicating the moisture content contained in a part approximately 15 µm inside from the skin surface of the forehead is shown.

Table 3

forehead average value

| (2) water | before intake | after 28 days | after 56 days |
|---|---|---|---|
| 1 | 46.73 | 46.03 | 46.50 |
| 2 | 48.79 | 47.33 | 47.43 |
| 3 | 49.49 | 47.79 | 48.10 |
| 4 | 49.59 | 47.98 | 48.12 |
| 5 | 49.60 | 49.45 | 48.88 |
| 6 | 52.37 | 51.77 | 52.95 |
| 7 | 56.52 | 53.37 | 54.41 |
| 8 | 61.37 | 59.85 | 58.87 |

Table 4 shows the quartiles of the test result.

Table 4

forehead average value

| (2) water | before intake | after 28 days | after 56 days |
|---|---|---|---|
| highest value | 61.37 | 61.37 | 58.87 |
| third quartile | 54.45 | 52.57 | 53.68 |
| second quartile | 49.60 | 48.72 | 48.5 |
| first quartile | 49.14 | 47.56 | 47.77 |
| lowest value | 46.73 | 46.03 | 46.03 |

(3) Table 5 shows the measurement results of eight examinees taking the composition containing only the high-amylose cornstarch. The results measured by using the above described measuring instrument before the intake, 28 days after the intake and 56 days after the intake are shown. Namely, the value (average value of ten times of measurement) indicating the moisture content contained in a part approximately 15 µm inside from the skin surface of the forehead is shown.

Table 5

forehead average value

| (3) high-amylose cornstarch | before intake | after 28 days | after 56 days |
|---|---|---|---|
| 1 | 42.68 | 43.00 | 49.19 |
| 2 | 43.92 | 45.06 | 49.80 |
| 3 | 44.55 | 46.83 | 50.32 |
| 4 | 45.31 | 56.94 | 57.80 |
| 5 | 50.29 | 59.61 | 57.95 |
| 6 | 51.90 | 59.70 | 62.77 |
| 7 | 54.37 | 62.19 | 65.07 |
| 8 | 64.85 | 64.16 | 76.92 |

Table 6 shows the quartiles of the test result.

Table 6

forehead average value

| (3) high-amylose cornstarch | before intake | after 28 days | after 56 days |
|---|---|---|---|
| highest value | 64.85 | 64.16 | 76.92 |
| third quartile | 53.14 | 60.95 | 63.92 |
| second quartile | 47.80 | 58.28 | 57.88 |
| first quartile | 44.24 | 45.95 | 50.06 |
| lowest value | 42.68 | 43.00 | 49.19 |

(4) Table 7 shows the measurement results of eight examinees taking the composition containing the high-amylose cornstarch and the inulin. The results measured by using the above described measuring instrument before the intake, 28 days after the intake and 56 days after the intake are shown. Namely, the value (average value of ten times of measurement) indicating the moisture content contained in a part approximately 15 µm inside from the skin surface of the forehead is shown.

Table 7

forehead average value

| (4) high-amylose cornstarch + inulin | before intake | after 28 days | after 56 days |
|---|---|---|---|
| 1 | 40.18 | 48.82 | 52.79 |
| 2 | 53.92 | 52.14 | 55.44 |
| 3 | 40.18 | 47.79 | 52.94 |
| 4 | 52.42 | 55.02 | 54.63 |
| 5 | 41.37 | 43.29 | 45.44 |
| 6 | 44.16 | 51.45 | 56.14 |
| 7 | 40.59 | 40.76 | 40.48 |
| 8 | 48.28 | 55.49 | 58.91 |

Table 8 shows the quartiles of the test result.

Table 8

forehead average value

| (4) high-amylose cornstarch + inulin | before intake | after 28 days | after 56 days |
|---|---|---|---|
| highest value | 53.92 | 55.49 | 58.91 |
| third quartile | 50.35 | 53.58 | 55.79 |
| second quartile | 42.77 | 50.14 | 53.79 |
| first quartile | 40.39 | 45.54 | 49.12 |
| lowest value | 40.18 | 40.76 | 40.48 |

(5) Table 9 shows the measurement results of eight examinees taking the composition containing six kinds of components. The results measured by using the above described measuring instrument before the intake, 28 days after the intake and 56 days after the intake are shown. Namely, the value (average value of ten times of measurement) indicating the moisture content contained in a part approximately 15 µm inside from the skin surface of the forehead is shown.

Table 9

forehead average value

| (5) six kinds of components | before intake | after 28 days | after 56 days |
|---|---|---|---|
| 1 | 41.36 | 44.42 | 52.78 |
| 2 | 48.84 | 54.08 | 58.07 |
| 3 | 53.49 | 58.36 | 60.07 |
| 4 | 54.82 | 58.54 | 64.18 |
| 5 | 54.95 | 60.15 | 69.58 |
| 6 | 55.67 | 66.66 | 71.13 |
| 7 | 56.64 | 67.13 | 75.82 |
| 8 | 56.91 | 67.56 | 83.71 |

Table 10 shows the quartiles of the test result.

Table 10

forehead average value

| (5) six kinds of components | before intake | after 28 days | after 56 days |
|---|---|---|---|
| highest value | 56.91 | 67.56 | 83.71 |
| third quartile | 56.16 | 66.90 | 73.48 |
| second quartile | 54.89 | 59.35 | 66.88 |
| first quartile | 51.17 | 56.22 | 59.07 |
| lowest value | 41.36 | 44.42 | 52.78 |

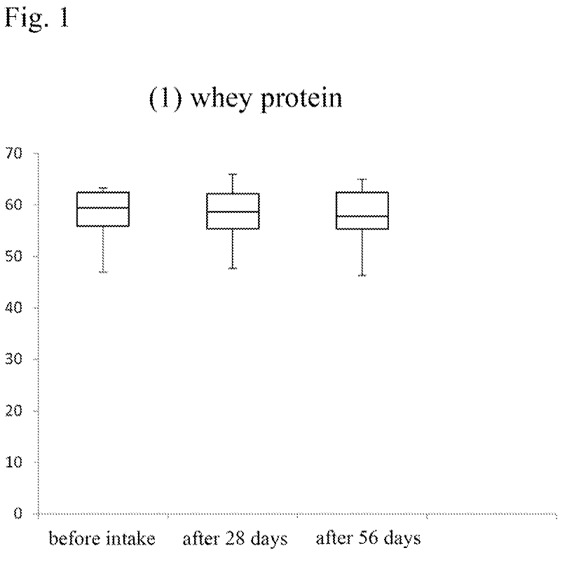

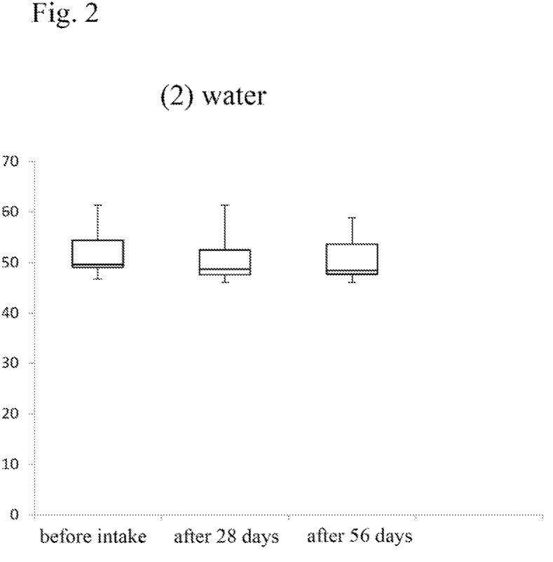

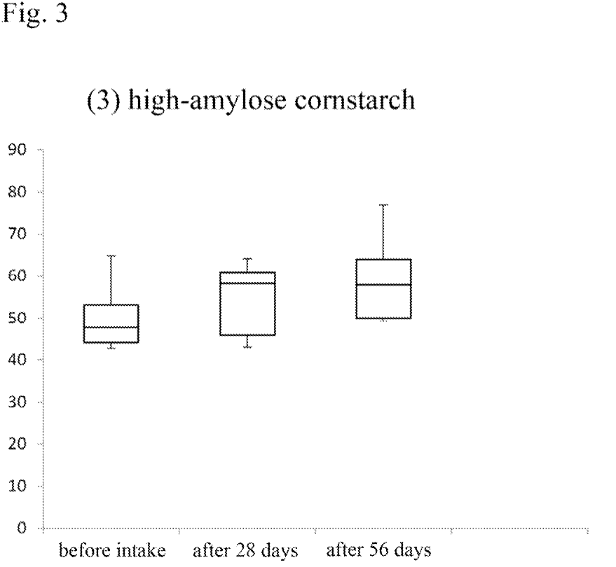

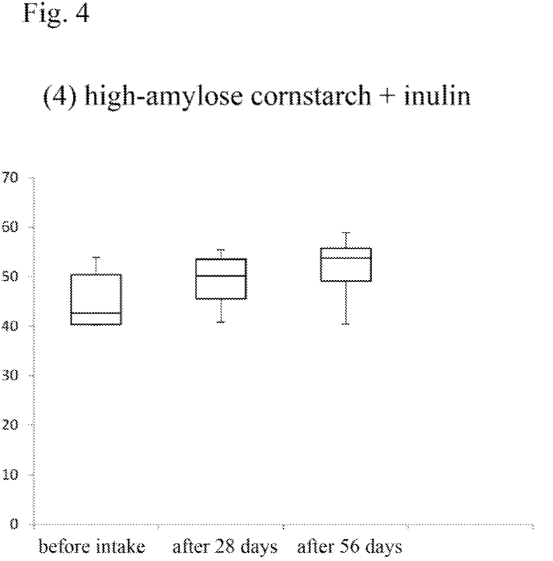

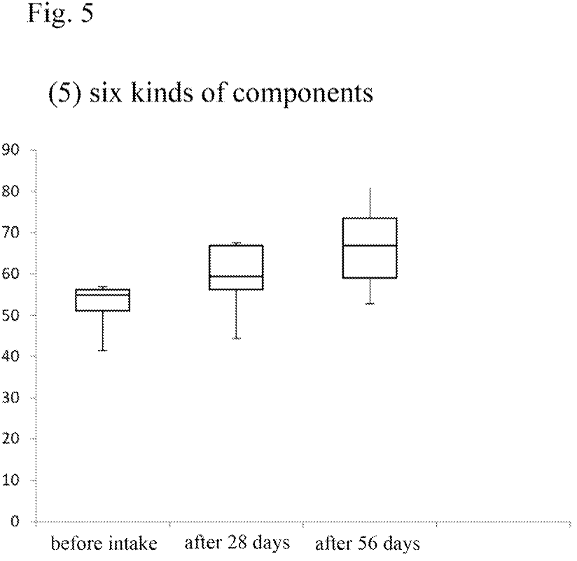

FIGS. 1 to 5 show the test results using the graphs of box plot type. The box plot shows the distribution of data using the quartiles. FIG. 1 is the test result of eight examinees taking (1) the whey protein. FIG. 2 is the test result of eight examinees taking (2) water. FIG. 3 is the test result of eight examinees taking (3) the composition containing only the high-amylose cornstarch. FIG. 4 is the test result of eight examinees taking (4) the composition containing the high-amylose cornstarch and the inulin. FIG. 5 is the test result of eight examinees taking (5) the composition containing six kinds of components.

In the test results of eight examinees taking (1) the whey protein which is the placebo drug and (2) eight examinees taking the water which is the placebo drug, the significant change of the moisture content could not be seen before the intake, 28 days after the intake and 56 days after the intake (shown in FIG. 1 and FIG. 2).

In an embodiment of the composition of the present invention which is (3) the composition containing only the high-amylose cornstarch, the significant change of the moisture content could be seen in the third quartile and the second quartile when before the intake and 28 days after the intake were compared (shown in FIG. 3). In addition, the significant change of the moisture content could be seen in all of the highest value, the third quartile, the second quartile, the first quartile and the lowest value when before the intake and 56 days after the intake were compared (shown in FIG. 3). It can be confirmed that a so-called water capacity of the skin was increased.

In an embodiment of the composition of the present invention which is (4) the composition containing the high-amylose cornstarch and the inulin, the significant change of the moisture content could be seen in the third quartile and the second quartile when before the intake and 28 days after the intake were compared (shown in FIG. 4). In addition, the significant change of the moisture content could be seen in all of the highest value, the third quartile, the second quartile, the first quartile and the lowest value when before the intake and 56 days after the intake were compared (shown in FIG. 4). It can be confirmed that a so-called water capacity of the skin was increased.

In an embodiment of the composition of the present invention which is (5) the composition containing six kinds of components, the significant change of the moisture content could be seen in all of the highest value, the third quartile, the second quartile, the first quartile and the lowest value when before the intake and 28 days after the intake were compared (shown in FIG. 5). In addition, the significant change of the moisture content could be seen in all of the highest value, the third quartile, the second quartile, the first quartile and the lowest value when before the intake and 56 days after the intake were compared (shown in FIG. 5). Furthermore, the significant change of the moisture content could be seen in all of the highest value, the third quartile, the second quartile, the first quartile and the lowest value when 28 days after the intake and 56 days after the intake were compared (shown in FIG. 5). It can be confirmed that a so-called water capacity of the skin was increased.

From the test results shown above, it is suggested that (3) the composition containing only the high-amylose cornstarch, (4) the composition containing the high-amylose cornstarch and the inulin and (5) the composition containing six kinds of components have the effect of improving the moisture content of the skin (i.e., effect of improving skin properties).

Additionally, the impressions of the examinees in the test will be described below. Impressions of the examinees taking (3) the composition containing only the high-amylose cornstarch
Makeup adheres better.
Significant dryness of jaw disappeared.
Skin becomes moist.
Skin gloss is seemed to be improved.

Impressions of the examinees taking (5) the composition containing six kinds of components
Makeup adheres well.
People say skin texture is improved.
Skin condition is good.
Skin seems to be hardly dried.
A dried part disappeared.
Tension is felt on the skin.

The effect of improving skin properties is also suggested from the above described impressions of the examinees.

Impressions of the examinees taking (4) the composition containing the high-amylose cornstarch and the inulin

The impressions were same as the above described impressions of (3). In addition, the following improvement of the bowel motion was seen.

Before the intake, three of eight examinees had chronic constipation and the rest five defecated approximately once per day.

In the above described five, feces are relatively soft in one of them. There is nothing worthy of mention about the rest four.

The bowel motion of the examinees taking the composition containing the high-amylose cornstarch and the inulin was as follows 28 days after the intake and 56 days after the intake.

The bowel motion was confirmed almost every day for all of eight examinees. The number of the bowel motions was increased to twice or three times per day for six of them. The state of the feces was relatively soft and the bowel motion was smooth.

What is claimed is:

1. A composition for improving skin properties, the composition comprising:
a high-amylose cornstarch.

2. The composition according to claim 1, further comprising:
an inulin.

3. The composition according to claim 1, further comprising:
a fructo-oligosaccharide.

4. The composition according to claim 2, further comprising:
a β-glucan.

5. The composition according to claim 4, further comprising:
a lactic acid bacterium.

6. The composition according to claim 1, further comprising:
a soybean.

7. The composition according to claim 1, wherein
the high-amylose cornstarch contains 60% or more of a moist heat-treated resistant starch.

8. The composition according to claim 1, wherein
an amount of the composition is 15 g or more.

9. The composition according to claim 1, wherein
an amount of the composition is four weeks’ worth or more.

10. A method for improving skin properties, the method comprising:
a step of taking a high-amylose cornstarch by a daily amount effective for improving the skin properties for four weeks or more, wherein
the method excludes a medical treatment to human.
